(19) United States
(12) Patent Application Publication
(10) Pub. No.: US 2023/0104483 A1
(43) Pub. Date: Apr. 6, 2023

(54) ROTATING MRI COILS FOR SAFE IMAGING OF PATIENTS WITH ELECTRONIC IMPLANTS

(71) Applicant: Northwestern University, Evanston, IL (US)

(72) Inventor: Laleh Golestani Rad, Wilmette, IL (US)

(21) Appl. No.: 17/959,674

(22) Filed: Oct. 4, 2022

Related U.S. Application Data
(60) Provisional application No. 63/252,381, filed on Oct. 5, 2021.

(51) Int. Cl. G01R 33/341; A61B 5/055

(52) U.S. Cl. CPC G01R 33/341; A61B 5/055; G01R 33/3628

(57) ABSTRACT

A system to perform magnetic resonance imaging includes a transmit coil that has a plurality of transmitters. The transmit coil is configured to receive at least a portion of an implant that is within a pediatric patient. The system also includes a controller operatively coupled to the transmit coil. The controller is configured to identify a region within the transmit coil with zero electric field while the transmitters are transmitting. The controller is also configured to rotate the transmit coil around the pediatric patient such that the implant is located within the region with zero electric field to avoid radio frequency heating of the implant.

### CROSS-REFERENCE TO RELATED APPLICATION

The present application claims the priority benefit of U.S. Provisional Patent App. No. 63/252,381 filed on Oct. 5, 2021, the entire disclosure of which is incorporated by reference herein.

### BACKGROUND

Medical resonance imaging, or MRI, refers to a non-invasive imaging technique in which an MRI system captures images of a patient's organs, tissue, skeletal system, etc. An MRI system utilizes powerful magnets, along with radio waves, and a computer processing system to generate detailed images of a patient. The radio waves used during an MRI procedure can result in radio frequency (RF) heating. RF heating occurs when high frequency radio waves interact with an electrical conductor, such as various metals and other materials. Specifically, change in the RF waves create a changing electromagnetic field in the electrical conductor, which results in molecular movement that generates heat.

### SUMMARY

An illustrative system to perform magnetic resonance imaging includes a transmit coil that has a plurality of transmitters. The transmit coil is configured to receive at least a portion of an implant that is within a pediatric patient. The system also includes a controller operatively coupled to the transmit coil. The controller is configured to identify a region within the transmit coil with zero electric field while the transmitters are transmitting. The controller is also configured to rotate the transmit coil around the pediatric patient such that the implant is located within the region with zero electric field to avoid radio frequency heating of the implant.

In an illustrative embodiment, the plurality of transmitters are linearly polarized. In some embodiments, the controller is further configured to determine, based on patient-specific information regarding a location of the implant within the pediatric patient, an optimal initial angle for the transmit coil. In another embodiment, the transmit coil is in the form of a birdcage coil that includes a plurality of ribs, and the transmitters are mounted on the ribs. In another embodiment, the controller is configured to identify a plurality of angles of the transmit coil that result in the region with zero electric field.

The system can also include an adjustable head array that has a plurality of receivers and an adjustable head cradle that is configured to accommodate pediatric patients with varying head sizes. In one embodiment, the plurality of receivers are formed from copper wires, and the receivers receive signals from the plurality of transmitters in the transmit coil. In another embodiment, each of the copper wires includes one or more gaps cut therein to provide distributed capacitance. In another embodiment, the receivers are tiled in the adjustable head array as an array of overlapping loops. In one embodiment, the adjustable head array includes a capacitive bridge to couple power out of the adjustable head array through critical coupling at a desired resistance. In another embodiment, the adjustable head array includes a preamplifier to reduce coupling between neighboring receivers in the adjustable head array.

The system can also include a blanket array that has a plurality of receive elements, where the blanket array is sized to cover a front portion of the pediatric patient. In an illustrative embodiment, the receive elements are positioned in an overlapping arrangement within the blanket array. In another embodiment, the receive elements are formed from flexible coaxial cable. The blanket array can be formed from one or more textiles, and the one or more textiles can be arranged to include a plurality of pockets to receive the plurality of receive elements. The blanket array can also include a printed circuit board positioned between layers of the one or more textiles. The blanket array can further include a protective pad positioned between the printed circuit board and the pediatric patient.

An illustrative method of performing magnetic resonance imaging includes positioning a transmit coil that includes a plurality of transmitters about a pediatric patient such that the transmit coil receives at least a portion of an implant that is within the pediatric patient. The method also includes identifying, by a controller, a region within the transmit coil with zero electric field while the transmitters are transmitting. The method further includes rotating, by the controller, the transmit coil around the pediatric patient such that the implant is located within the region with zero electric field to avoid radio frequency heating of the implant. In another embodiment, the method includes identifying, by the controller, a plurality of angles of the transmit coil that result in the region with zero electric field. In one embodiment, the method includes determining, by the controller and based on patient-specific information regarding a location of the implant within the pediatric patient, an optimal initial angle for the transmit coil.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts how an epicardial lead in an infant (left) has a completely different trajectory than an endocardial lead in an adult (right) in accordance with an illustrative embodiment.

FIG. 2 is a table that depicts example MRI protocols that cannot be substituted by other imaging tests in cardiac implantable electronic devices (CIED) patients in accordance with an illustrative embodiment.

FIG. 3A depicts an x-ray of a 4 year old pediatric patient treated with VNS for refractory epilepsy in accordance with an illustrative embodiment.

FIG. 3B depicts an MRI exclusion zone in accordance with an illustrative embodiment.

FIG. 3C depicts preliminary data comparing the 0.1 g-averaged SAR generated by a standard 1.5 T body coil (in CP mode) and an optimally adjusted rotating LP coil when the input power of both coils is adjusted to generate a B₁⁺=2 μT at the iso-center in accordance with an illustrative embodiment.

FIG. 4A includes EM simulations with a rotating head coil to demonstrate substantially reduced SAR when the Zero-E field region of the coil was aligned to co-inside with the implant in accordance with an illustrative embodiment.

FIG. 4B depicts the result of experiments conducted with a proof-of-concept rotating head coil developed in lab to confirm substantially reduction of RF heating around implanted deep brain stimulation leads in a head phantom in accordance with an illustrative embodiment.

FIG. 5A depicts the position of zero-E regions during body imaging in accordance with an illustrative embodiment.

FIG. 5B depicts the 0.1 g-averaged SAR in accordance with an illustrative embodiment.

FIG. 5C shows rotation angles of the coil in accordance with an illustrative embodiment.

FIG. 6 is a 3D illustration of the RPA pediatric coil system including a rotating transmitter coil, a 32-channel size adjustable head coil, a posterior cradle which houses 16 coil elements, and a flexible 16-channel blanket coil array in accordance with an illustrative embodiment.

FIG. 7A is a simulation of a rotating head coil around a head phantom with a deep brain stimulation implant in accordance with an illustrative embodiment.

FIG. 7B is an image-based metric to quantify the visual distortion in B₁⁺ maps (line with stars) perfectly correlated with the SAR amplification at a lead's tip in accordance with an illustrative embodiment.

FIG. 8 is a diagram that depicts the proposed RPA imaging workflow in accordance with an illustrative embodiment.

FIG. 9 is a block diagram of the proposed system in accordance with an illustrative embodiment.

### DETAILED DESCRIPTION

Magnetic resonance imaging (MRI) has revolutionized the approach to understanding human biology and pathology. Three decades after the first image of a live human was produced, MRI was listed as one of the top three breakthroughs that changed the history of medicine. Unfortunately, despite continuous advances MRI remains inaccessible to a large and growing group of patients with conductive implants. The problem is exacerbated in children, for whom MR-conditional devices are not readily available. Additionally, manufacturers are less willing to invest in such technology for children as the pediatric market is smaller than the adult market.

The major risk of MRI in patients with conductive implants is the radio frequency (RF) heating of the tissue due to the antenna effect. This occurs when the electric field of an MRI scanner couples with the implanted device and amplifies the specific absorption rate (SAR) of the radiofrequency energy in the tissue surrounding the implant. Serious injuries have underscored the severity of this problem. MR-conditional implants (such as cardiovascular electronic devices) have been approved by the FDA for adults, allowing patients to receive MRI under restricted conditions that assure the safety of the patient. Regrettably, however, neither MRI vendors nor device manufacturers have established safe MRI methodologies for children with conductive implants. This leaves a vulnerable population unable to receive the standard of care that they need the most and excludes them from scientific inquiries when they are most advantageous.

There is a steady growth in the use of medical implantable devices in the US and globally, with children constituting a growing portion of device recipients. The pediatric medical device market is projected to grow by 10% annually, reaching $54 billion by 2028. The trend is likely to accelerate due to recent regulatory initiatives and paternal awareness programs. Due to its excellent soft tissue contrast and non-invasive nature, magnetic resonance imaging (MRI) has become the modality of choice for a majority of neurological, cardiac, and musculoskeletal disorders. It is estimated that 75% of patients with medical devices will need to undergo an MRI scan during their lifetime, with many patients requiring repeated examinations. The number is likely higher in children, as developmental changes warrant more frequent assessment and alternative ionizing modalities are more restricted.

Until recently, MRI was contraindicated in patients with active medical implants due to risk of interactions between MRI fields and the implanted device. Since the first report of such harmful effects in 1989, significant improvement has been made in implant design and manufacturing to reduce the effects of static magnetic field and gradients. RF heating from excitation fields, however, remains a major issue. This antenna effect occurs when the electric field of the MRI transmit coil couples with an elongated conductive lead, causing the specific absorption rate (SAR) of the RF energy to amplify at the lead's tip. Fatal injuries have underscored the severity of this issue. MR-conditional devices have been developed for adults, allowing patients to receive MRI under restricted conditions that assure safety. The MR-Conditional labeling, however, does not easily transcend to pediatric populations as briefly outlined below.

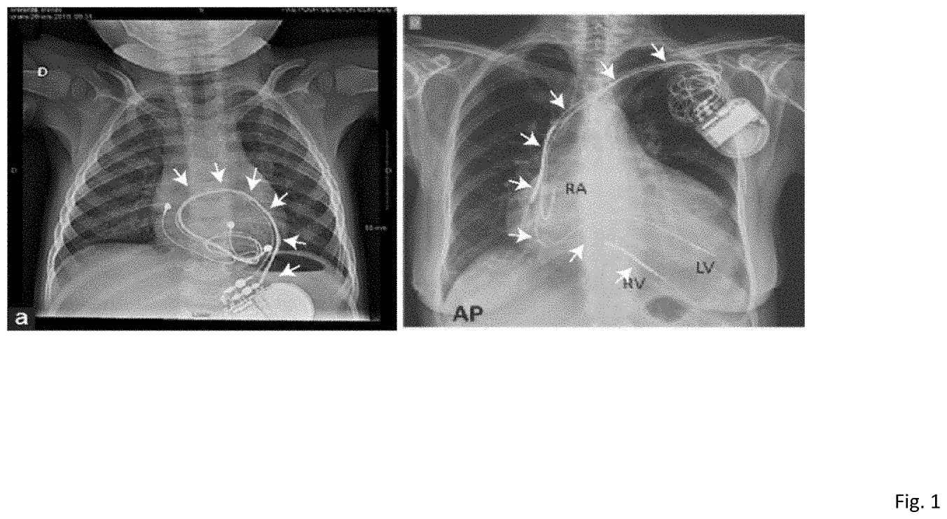

Infants and children with congenital heart defects, inherited arrhythmia syndromes, and/or congenital disorders of cardiac conduction often receive a cardiac implantable electronic device (CIED). Some infants receive a CIED within hours, or even minutes, of birth. Due to the small size and venous anatomy, the standard approach to affixing a CIED to the heart of a young patient is to open the chest and sew the cardiac lead directly to the myocardium (“epicardial leads”) as opposed to the adult model of passing it through veins and affixing it to the inside of the heart (“endocardial leads”). FIG. 1 depicts how an epicardial lead in an infant (left) has a completely different trajectory than an endocardial lead in an adult (right) in accordance with an illustrative embodiment. The trajectory of the epicardial lead (infant) leads to much higher RF heating.

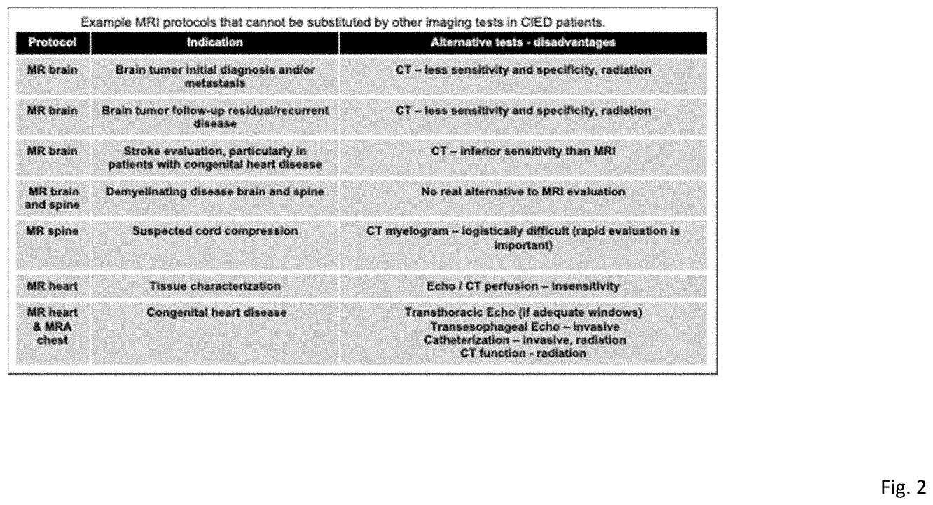

Unfortunately, once epicardial leads have been implanted, the patient is contraindicated for an MRI exam due to the elevated risk of RF heating at the tip of epicardial leads. The problem is exacerbated by the fact that there is no straightforward method to extract epicardial leads, so children who receive these leads are excluded from the benefits of MRI for life, even if a subsequent FDA-approved endocardial system is placed when they are older. Even more compellingly, this restriction is not unique to cardiac MRI. The presence of epicardial leads is a contraindication for MRI elsewhere in the body, preventing patients from receiving MRI for other injuries and diseases. Lack of access to MRI leads to suboptimal diagnosis, prognosis, and surgical planning as many MRI protocols cannot be substituted with CT, echocardiography, or catheterization with fluoroscopy. FIG. 2 is a table that depicts example MRI protocols that cannot be substituted by other imaging tests in cardiac implantable electronic devices (CIED) patients in accordance with an illustrative embodiment.

Other implants that can result in RF heating include vagus nerve stimulation devices and electrocorticography (EcoG) electrodes. Epilepsy affects 1% of the pediatric population in the US, yet its treatment costs $12 billion. For approximately 30% to 40% of children whose seizures fail to respond to antiepileptic drugs or ketogenic treatment, vagus nerve stimulation (VNS) is the only alternative. VNS has shown significant efficacy in the reduction of seizure frequency in young children (<5 years old), and is currently being explored as treatment for a variety of autoimmune diseases and chronic inflammatory conditions due to its demonstrated anti-inflammatory properties.

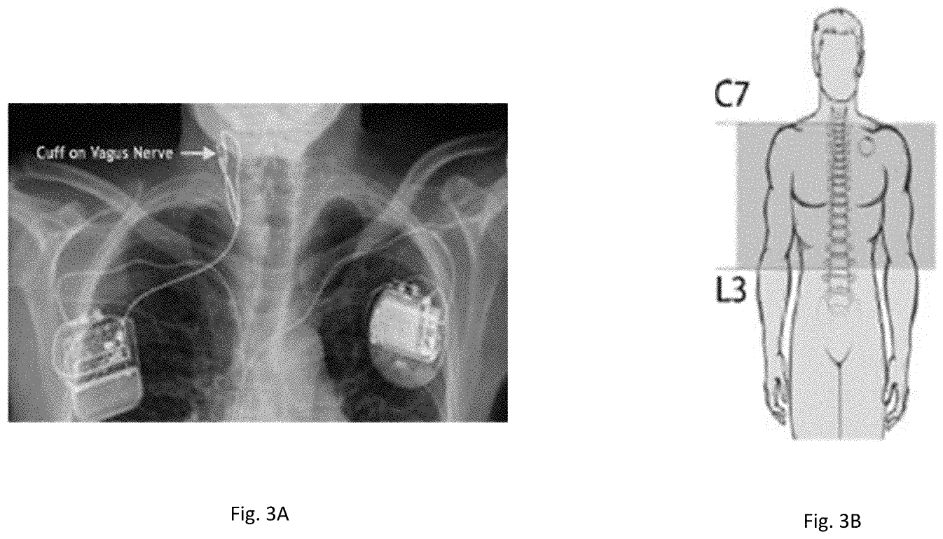

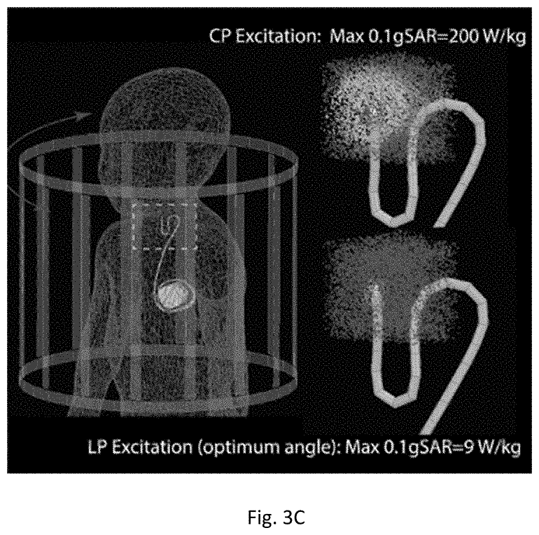

The current Livanova© (formerly Cyberonics) implantable treatment device includes a small battery-powered stimulator and a fine wire electrode that extends from the battery and is typically wrapped around the left cervical vagus. Unfortunately, once the device is implanted, a large area of the body (from L3 to C7) will be excluded from MRI. FIG. 3A depicts an x-ray of a 4 year old pediatric patient treated with VNS for refractory epilepsy in accordance with an illustrative embodiment. FIG. 3B depicts an MRI exclusion zone in accordance with an illustrative embodiment. FIG. 3C depicts preliminary data comparing the 0.1 g-averaged SAR generated by a standard 1.5 T body coil (in CP mode) and an optimally adjusted rotating LP coil when the input power of both coils is adjusted to generate a B₁⁺=2 μT at the iso-center in accordance with an illustrative embodiment. The preliminary studies show that the proposed rotating coil technology can reduce the 0.1 g-averaged SAR around the tip of VNS leads by 22-fold compared to the standard body coil, even when the MRI exclusion zone was at the coil's iso-center. Similarly, the simulations of high-density subdural EEG grids (Ad-Tech, Oak Creek, Wis.) implanted over the temporal lobe and motor cortex showed that a 20-fold reduction in SAR can be achieved using the rotating coil compared to the scanner's body coil. The preliminary data indicates that the proposed coil system will enable unrestricted body and brain MRI in children with VNS devices and EcoG electrodes.

The data also suggests that the coil system will perform equally well in children with other types of passive and active implants. This includes children with cardiac stents, abandoned leads, responsive neurostimulation (RNS) devices, and cochlear implants. Specifically, abandoned epicardial leads have been shown to generate RF heating as high as 60° during MRI. It is predicted that the proposed coil system will be in high demand across all pediatric subspecialties, as an additional important detail in children is the growth of lesions over time, making avoidance of RF heating evermore crucial.

Efforts to bring MRI accessible to patients with conductive implants has a long history. Most of the work has focused on modifying the implant itself, e.g., in the case of active implants by increasing the lead's impedance to reduce induced RF currents. This has had limited success and the restrictions on MRI remain. It has become clear that altering lead's design has a substantially high cost-to-benefit ratio for the manufacturer. For example, the safety margins gained are modest at best, whereas slight alterations in design or material trigger the long and costly process of re-obtaining FDA approval for biocompatibility, non-toxicity, and mechanical stability. On the other hand, modifying MRI hardware has the potential to massively improve safety margins in ways that are generally applicable to a wide range of devices, thus dramatically shifting the cost-benefit calculation.

Extensive efforts have been made to improve safety of MRI in patients with conductive implants. Yet, two decades of concerted effort have not solved the problem of implant imaging in children. Described herein is a system that fills this glaring gap in technology by bringing various innovations. In contrast to all previous attempts to improve compatibility through device modifications, the inventors propose to change the MR technology itself. This approach is absent from the literature. It is therefore believed that a fundamentally new approach is required to create MR compatibility via methodologies that are user-friendly in the clinical setting and also robust and versatile. To do this will involve a paradigm shift from computationally intensive algorithms to methods that are based on simple and quick mechanical maneuverings suitable for pediatric and other applications. As used herein, pediatric can be used to refer to patients that are neonates, infants, toddlers, and young children. Young children can refer to children that are age 8 or less, age 10 or less, age 12 or less, age 14 or less, etc., depending on the implementation.

The proposed system includes a reconfigurable patient-adjustable (RPA) pediatric coil system that can be plugged in to scanners like a standard local coil and operated by a technologist with minimal training. This will involve mechanically rotating the zero-E-field region of a linearly polarized birdcage transmitter such that implants, wherever they are located, are fully contained within it. In addition, a patient-specific pipeline is implemented to predetermine the initial position of the coil for each patient before they arrive at the MRI unit. One method to implement the patient-specific pipeline is to perform electromagnetic simulations on a representative group of patient-specific models of each device and determine the statistically optimum angle for that device/patient type. A user can then start the coil with this statistically optimal (but not individually fine-tuned) angle, and perform B₁ mapping to determine the individually optimized coil angle by minimizing the image artifact on the B₁ maps. As such, fine-tuning the coil to the optimum angle can be done on the fly (e.g., in less than ˜5 minutes) using a few low-SAR B₁ maps.

Integration of a dedicated rotating transmit body coil with age-optimized adjustable brain and body receiver arrays provides an ideal tool for pediatric imaging at 1.5 Tesla (T), which requires high sensitivity. A major advantage stems from the combination of the homogeneous excitation by the volume coil, the unique E-field dropout, and the high sensitivity of small surface coil detectors which allow the application of parallel acquisition methods such as SENSE, GRAPPA, and simultaneous multislice (SMS) accelerations. It is expected that the high SNR and acceleration factors enabled by a pediatric specific multichannel coil combined with the implant friendly excitation technology will make 5-minute brain scans and 10-minute cardiac/body scans possible in children with all kinds of devices, without the need for sedation or anesthesia.

While MRI hardware advancements have the potential to revolutionize pediatric imaging, accessibility challenges need to be addressed right at the early stage to maximize the impact. Thus, in an innovative departure from all previous approaches that are vendor specific, the inventors developed a multi-platform coil technology that can be plugged and played into scanners of two major MRI vendors that have combined market share of over 85% in the US. The approach to develop multi-platform coil technology with integrated head and body receive arrays promotes patient access and comfort in ways that have not been possible before.

Described herein is a novel multi-platform MRI coil technology that allows ultra-fast and high-resolution MRI to be safely performed in children with conductive implants regardless of the location of the implant within the body. The solution is based on the idea that through innovative engineering one can control local electric fields generated by MRI on a case-by-case basis, thus avoiding interactions with the individual's implanted device wherever it happens to be in the body. The preliminary results show that a linearly polarized birdcage transmitter has a slab-like region of low-electric field which can be mechanically rotated to coincide with an implanted device to virtually eliminate RF heating. It was found that this technique can achieve a 20-fold reduction in RF heating of all types of cardiac implantable electronic devices (CEIDs) and implants of the central nervous system including EcoG electrodes and Vagus Nerve Stimulation (VNS) devices. The device is also adjustable on a patient-by-patient basis such that it is effective on all patients. Such reduction in RF heating is sufficient to support all clinical and research sequences of interest in brain and body imaging. Additionally, the proposed technology provides a probe to study pathologies as they develop, evolve, and respond to the intervention, enabling acquisition of biomedical knowledge in ways that have not been possible before.

In an illustrative embodiment, the proposed system includes the following integrated parts. The system includes a patient-adjustable rotating MRI transmit coil that eliminates RF heating in children with implants of the cardiac and nervous system. The system also includes age-optimized close-fitting head and body receive coil arrays that integrate with the rotating transmitter to allow high-resolution imaging. The system also utilizes methods to determine the safe range of parameters that allow imaging of children with cardiac and central nervous system (CNS) devices with the ensembled coil system. Details of the proposed technology are included below.

As discussed above, the proposed system is based on modifying the electric field of an MRI transmit coil to minimize its interaction with conductive implants and reduce the risk of RF heating. It has been shown that by weighting the amplitude of the input channels of a dual-derived birdcage transmitter, the coil could be derived in a linear mode, generating a plane of zero electric field (zero-E field) which could then be steered to coincide with the location of a wire implant to reduce RF heating. However, calculating the input signals necessary to create and steer the zero-E field region is so complex and time consuming that it effectively impedes clinical application. With the advancement of multi-channel transceiver coils and parallel transmit (pTx) technology, the concept of field-shaping for implant imaging was revisited. The inventors have investigated pTx for implant imaging whereby the input voltages of each transmit channel are weighted by a static complex factor and field maps are optimized to: (1) minimize the electric field within regions of interest (ROIs) along the length of a wire implant, while (2) maximizing the homogeneity of B₁⁺ on planes of interest (POIs). By defining a cost function that accounts for challenging scenarios, such as curved wire geometries and multiple implanted wires, it has been shown that RF heating of bilateral DBS leads with realistic trajectories could be reduced by 80% using an 8-element pTx system. Mathematically, the cost function is:

$${min}_{{A}_{n} , {φ}_{n}} ( \frac{{σ}_{POI} ( \vert {B}_{1 , tot}^{+} \vert )}{{μ}_{POI} ( \vert {B}_{1 , tot}^{+} \vert )} + λ \cdot {μ}_{R O M s} ( \frac{\vert {E}_{tot}^{( R O M s )} \vert}{\vert {E}_{q u a d}^{( R O M s )} \vert} ) ) , \qquad Equation 1$$

$$and {E}_{( . )}^{( R O M s )} = [ {E}_{( . )}^{( 1 )} {E}_{( . )}^{( 2 )} {E}_{( . )}^{( 3 )} … {E}_{( . )}^{( M )} ] ,$$

where σ_{POI}(⋅) represents the standard deviation over the POI, μ_{POI}(⋅) represents the mean over the POI; μ_{POI}(⋅) represents the mean over the ROMs; and λ is a weighting factor to determine the tradeoff between safety (minimum E-field) and image quality (homogenous B₁⁺).

Although the SAR reduction was promising, pTx has drawbacks for clinical applications. First, optimizing and implementing signal weights is complex as it requires substantial expertise and high-level access to the scanner hardware that is not easily available in clinical settings. Second, B₁⁺ homogeneity cannot be effectively controlled by shimming alone and remains as high as 40% in many cases. Using “dynamic” pTx with fully independent signals can potentially improve the problem (at the expense of adding multi-channel amplifiers and their energy chain to the scanner), however, the new preliminary data shows that even at its best theoretical implementation, the performance of pTx is not superior to the rotating coil technology described herein.

The proposed system is a plug-and-play, easy to operate solution for implant-friendly pediatric MRI based on the concept of reconfigurable patient-adjustable (RPA) coil technology. The idea is that instead of manipulating the input signals of a stationary coil to create and steer a zero-E field region, one can simply use a linearly polarized transmitter and mechanically rotate it around the patient's body such that its inherent zero-E field region coincides with the individual's implant. As used herein, a zero-E field region can include small electric fields, such as electric fields that are less than 10% of the maximum field generated by the coil in the sample.

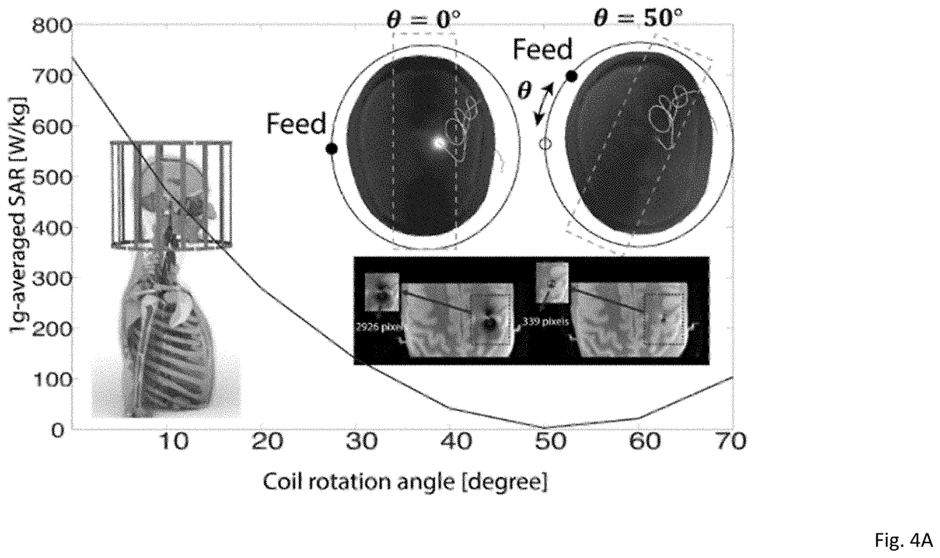

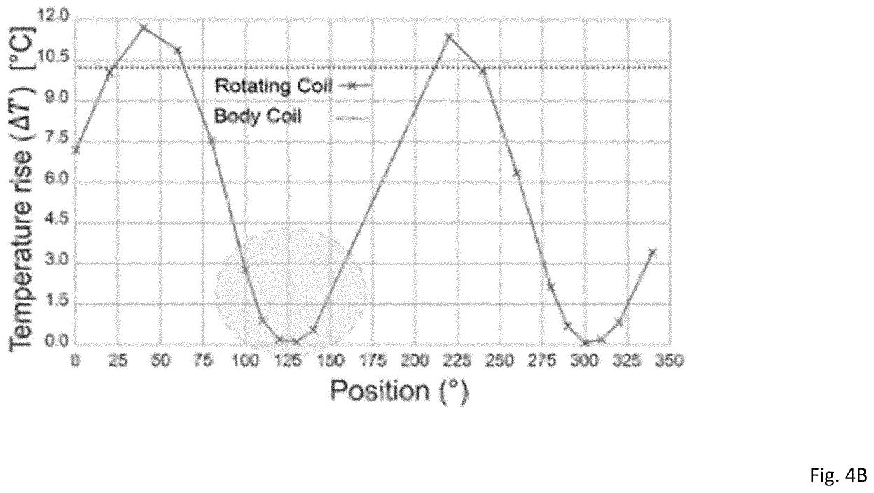

FIG. 4 shows this concept explored in prior work for imaging patients with deep brain stimulation (DBS) implants. This technique has a major advantage for pediatric imaging, as operating an RPA coil is substantially easier than operating a pTx system or a dual-derived body coil, a crucial aspect for imaging an inherently uncooperative patient population. Specifically, in prior work, the inventors applied an image segmentation and reconstruction pipeline to CT images of a patient with a deep brain stimulation implant to create a realistic 3D model for finite element simulations. FIG. 4A includes EM simulations with a rotating head coil to demonstrate substantially reduced SAR when the Zero-E field region of the coil was aligned to co-inside with the implant in accordance with an illustrative embodiment. The non-susceptibility artifact around the DBS lead tip due to B₁⁺ distortion was also substantially reduced as demonstrated in a cadaveric brain. FIG. 4B depicts the result of experiments conducted with a proof-of-concept rotating head coil developed in lab to confirm substantially reduction of RF heating around implanted deep brain stimulation leads in a head phantom in accordance with an illustrative embodiment. The highlighted area of FIG. 4B indicates the deviation from the optimal angle, for which the RF heating generated by the rotating coil remained within 25% of the RF heating generated by the standard body coil. As it can be seen from the figure, deviations as large as 40° around the optimal angel still produced RF heating at a fraction of the standard body coil.

The inventors have also shown that a rotating head coil at 127 MHz (3 T) can reduce the SAR around tips of bilateral DBS leads in adults by 95%. Preliminary studies find that this concept can be translated to pediatric full-body imaging at 1.5 T, where a scaled and retuned replica of the rotating coil can be redesigned such that it accommodates pediatric body sizes from infancy to 6 years of age (e.g., max shoulder-to-shoulder width of 8 inches). Alternatively, different sizes may be used. Specifically, the simulations show that such an RPA coil can reduce the 0.1 g-averaged SAR around tips of both epicardial and endocardial CIEDs by an average of 10-fold. This reduction in SAR will reduce the typical 7° C.-20° C. RF heating of epicardial leads to less than 2° C., allowing a wide range of MRI sequences to be safely applied. Similarly, simulations with a vagus nerve stimulator (VNS Therapy, Houston, Tex., PerenniaFLEX Lead Model 304) showed a 15-25-fold reduction in SAR when the body model was positioned within the MRI coil at imaging landmarks covering neck to lower abdomen (C7-L3), a region that is currently an exclusion zone for MRI. Finally, the simulation of a high-density subdural EEG grids (Ad-Tech, Oak Creek, Wis.) implanted over the temporal lobe and motor cortex showed a 20-fold reduction in SAR achieved by the RPA coil.

The inventors validated the RPA transmit coil in part by creating three cohorts of pediatric body models with realistic representations of each device type. Simulations were then performed to determine the statistically optimum coil angle for each device type. The inventors designed and constructed the rotating transmitter to allow at least 50 degree rotation around each predetermined optimal angle. Additionally, the inventors constructed pediatric anthropomorphic phantoms, implanted them with CIEDs, EcoG electrodes, and VNS devises, performed RF heating experiments, and determined the heating reduction performance of the coil in practice.

Regarding the rotating transmitter, a linearly polarized low-pass birdcage transmitter was designed to be used in 1.5 T Siemens MAGNETOM and 1.5 T GE Singa MRI platforms. In alternative embodiments, different MRI platforms may be used. The mechanical housing for the transmitter allows smooth rotation of the coil around its axis with high precision (<5° increments) and a user-controlled dynamic rotation range of −50°<X°<50° around six predetermined feed positions in the azimuthal plane, namely, θ_{1,h/b}, θ_{2,h/b} and θ_{3,h/b} such that X° rotation around each predetermined position (i.e., θϵ[θ_{i,h/b}−X°, θ_{i,h/b}+X°]i=1, 2, 3) minimizes the SAR at tips of CIED leads, EcoG grid electrodes, and VNS leads, during imaging at head (h) or body (b) landmarks, respectively. Alternatively, a different rotation range (e.g., +/−45°, +/−60°, +/−70°, etc.) and/or a different rotation increment (e.g., 3°, 4°, 6°, etc.) may be used. Similarly, a different number of predetermined feed positions may be used, such as 5, 7, 8, 10, etc. To determine each θ_{i,h/b} finite element simulations were performed with patient-derived models (i.e., 30 CIEDs, 15 EcoG systems, 15 VNS devices). From these simulations, the optimum angles θ that maximize a metric defined as the SAR-reduction efficiency (SRE) of the coil for each device category can be determined using Equation 2:

$$S R {E}_{n} ( θ ) = 100 \frac{Max 1 g {SAR}_{CP , n} - Max 1 gSA {R ( θ )}_{R P A , n}}{Max 1 gSA {R}_{C P , n}} \qquad Equation 2$$

In Equation 2, Max1gSAR_{CP,n} and Max1gSAR(θ)_{RPA,n} are the maximum 1 g-averaged SAR at the tip of device lead/electrode n generated by a quadrature body coil and the rotating coil at position θ, respectively, when the input power of both coils is adjusted to produce the same flip angle (i.e., angle at which the axis of the hydrogen proton shifts from its longitudinal plane (static magnetic field B₀) Z axis to its transverse plane XY axis by excitation with the help of radiofrequency (RF) pulses) on a POI. The predetermined positions for each device category can be set at the average of optimal angles for that category θ_{i,h/b}=θ̅ₒₚₜ.

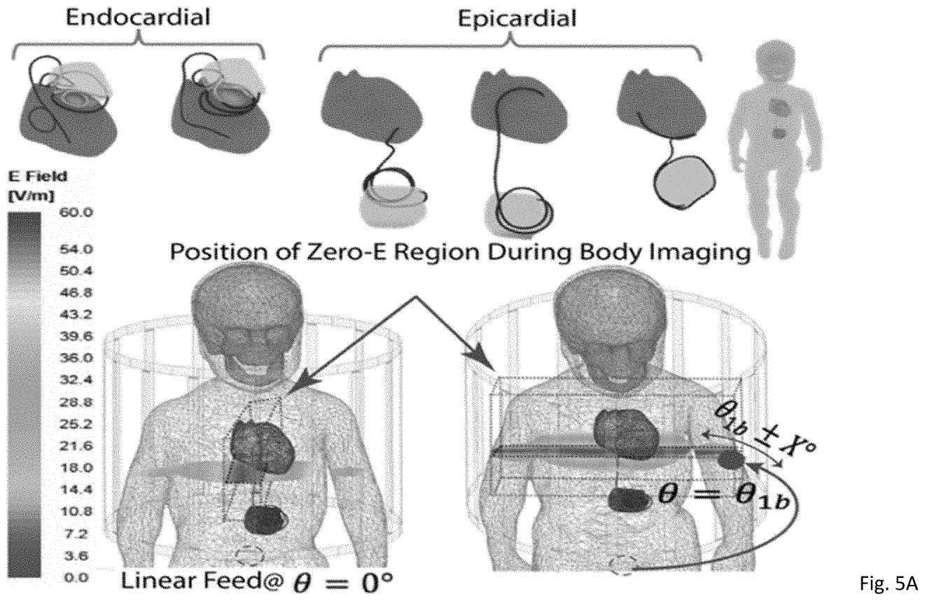

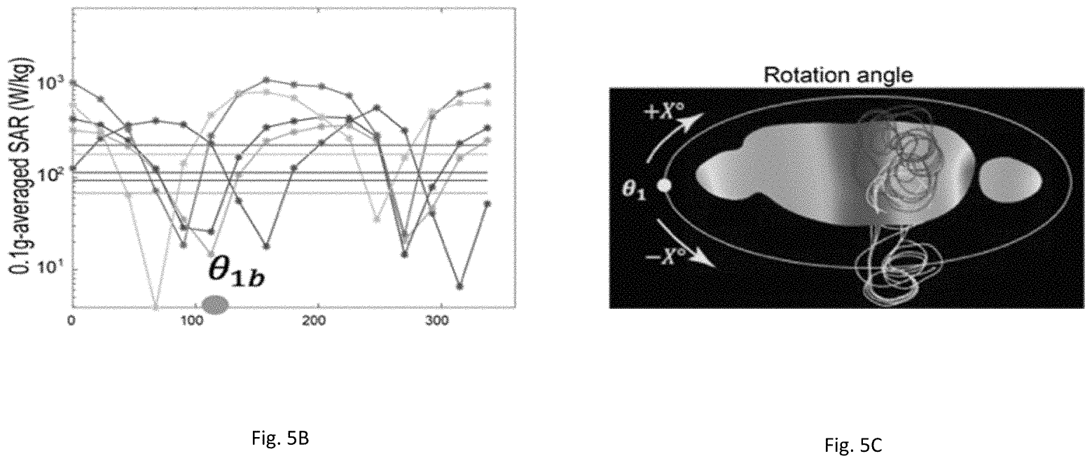

FIG. 5 is based on models of epicardial and endocardial CIEDs that were created from postop CT images and registered to a body model based on images of a 29-month-old patient. The 0.1 g-averaged local SAR created by the RPA coil during body imaging was simulated and compared to that of standard body coil when the input power of both coils was adjusted to generate the same B₁⁺ at the iso-center. For all cases, the inventors were able to find an optimum coil angle that reduced the SAR by >98%. FIG. 5A depicts the position of zero-E regions during body imaging in accordance with an illustrative embodiment. FIG. 5B depicts the 0.1 g-averaged SAR in accordance with an illustrative embodiment. FIG. 5C shows rotation angles of the coil in accordance with an illustrative embodiment. This experiment can be repeated with (e.g., N=30) implant and body models to determine angles θ₁ₕ and θ_{1b} that statistically minimize SAR in patients with CIEDs during head and body imaging, respectively. The coil mechanical housing can be designed to allow ±X=50° rotation around θ₁ₕ and θ_{1b} to enable fine tuning RF heating for each individual during head or body imaging. In alternative embodiments, an angle other than 50° may be used, such as 45°, 55°, 60°, 70°, etc.

It has been shown that a coil set of sized-optimized bespoke pediatric head array coils greatly enhance SNR and image encoding speed. The proposed infant MR imaging approach has addressed multiple aspects of coil array technology to substantially improve sensitivity, spatial resolution, encoding speed, motion insensitivity, and subject tolerance. Specifically, the inventors explored the concept of an adjustable coil array for awake task-based infant brain imaging and demonstrated SNR gains of 3-fold in cortical brain regions compared to a 32-ch adult array. Fast image encoding with multiband factor of 4 and in-plane acceleration of R=2 were feasible, and fMRI scan completion rates improved from <5% to 70% in awake infant scans.

Described below are methods to exploit the improved SNR and parallelism to accelerate image encoding, minimize the total acquisition time, and provide flexibility to collect a larger number of shorter scans. Most importantly, it is believed that neither sedation nor anesthesia will be necessary to complete infant MRI scans using the RPA technology, thus highly improving comfort and promoting access.

It is well established that tight-fitting arrays boost the sensitivity and speed of imaging. However, especially for infants, MRI coil detectors are better to be flexible to adapt to individual anatomy and follow posture. Two different sized 32-channel helmet receive arrays were constructed and validated for integration with a 1.5 T RPA coil using an adjustable head coil design. For pediatric body imaging, a posterior rigid coil array base was constructed, including 16 Rx elements, paired with two differently sized flexible coil blankets. The coil blanket includes 16 channels each with highly flexible Rx elements. The wearable array offers high sensitivity along with patient comfort, and considerably enhances the workflow as it can be put on quite easily, reducing the setup times.

Head coil design was based on the head and body circumference statistics for children. The inventors decided to break down the array size into age groups and suggest that sex differences in circumference for the 50% percentile are smaller than the variation between the 5^{th} and 95^{th} percentile in the group. For example, for 12-month-olds, the difference between the sexes in average circumference is 1.6 cm while the variation between the 5^{th} and 95^{th} percentile is 4.2 cm for both boys and girls. For the younger of the two groups, the circumference differences between the youngest and oldest within a given group also exceed the sex differences. The choice of 2 size-adjustable coil helmets with target ages of 0 to 2-year-old and 2 to 6-year-old was thus chosen based on the head circumference statistics.

Receiver coil arrays for the head are tiled with an array of overlapping loops. For the helmet design and the posterior body array, the receive coil elements can be constructed from copper wires with gaps cut for distributed capacitance. A capacitive bridge can be introduced to couple power out of the coil through critical coupling at 50Ω. The matching capacitor will additionally serve as part of an LC PIN diode trap circuit, designed to detune each receiver coil element during transmit. The pre-adjustment of this trap circuit can be carried out with a small inductive “sniffer” probe under an S₁₁ measurement, while the PIN diode is forward biased (i.e., transmit state, trap active). After all coil elements are assembled, this operation can be revisited for fine tuning the active detuning trap, starting with all the array elements switched off (i.e., detuning active). In such an embodiment, only the loop element under test is toggled on and off, while the active detuning is monitored using a double inductive probe kit with an S₁₂ measurement on the vector network analyzer. An active detuning of >35 decibels (dB) is going to be expected between these two states.

Preamplifier decoupling can be used to reduce coupling between next-nearest and further neighbors. Optimization of the preamplifier decoupling is a critical operation in constructing highly parallel arrays. The goal is to design the preamplifier/coil circuit so that the preamplifier performs a voltage measurement across the loop. Thus, the output circuitry (match+coax+preamplifier) forms a series high impedance in the tuned loop reducing current flow and thus reducing inductive coupling with other loop elements. To transform the preamplifier input impedance to a high impedance in the loop, it can be first transformed to a low impedance (short-circuit) across a parallel LC circuit tuned to the Larmor frequency, which in turn introduces a high serial impedance in the coil loop. In this mode, minimal current flows in the loop and inductive coupling to other coils are minimized.

Regarding blanket coil design, the exact sizing of the flexible body coil blankets is less critical due to the 2D surface structure and the high degree of flexibility to wrap it around the infant. The inventors designed and constructed two blanket coil arrays having a size of 30×40 cm and 50×60 cm. A stretchable textile made from 80% polyester and 20% elastane can be used to build the 16-channel wearable arrays. Alternatively, different sizes and/or materials may be used. Two layers of the elastic textile can be sewn together to create textile casings (or pockets) for embedding coil elements. The casings can be made wide enough for the coil conductors to be bent and relaxed evenly with little friction. The 16 coil receive-only elements in each blanket can be constructed from 1.8 mm diameter flexible coaxial cable (e.g., G 01132-06, Huber+Suhner, Herisau, Switzerland) and tiled in a 4×4 overlapped arrangement. This array geometry was chosen for balanced coverage, while limiting exposure of the coaxial element conductors to high curvature. In alternative embodiments, different configurations of the blanket coil can be used.

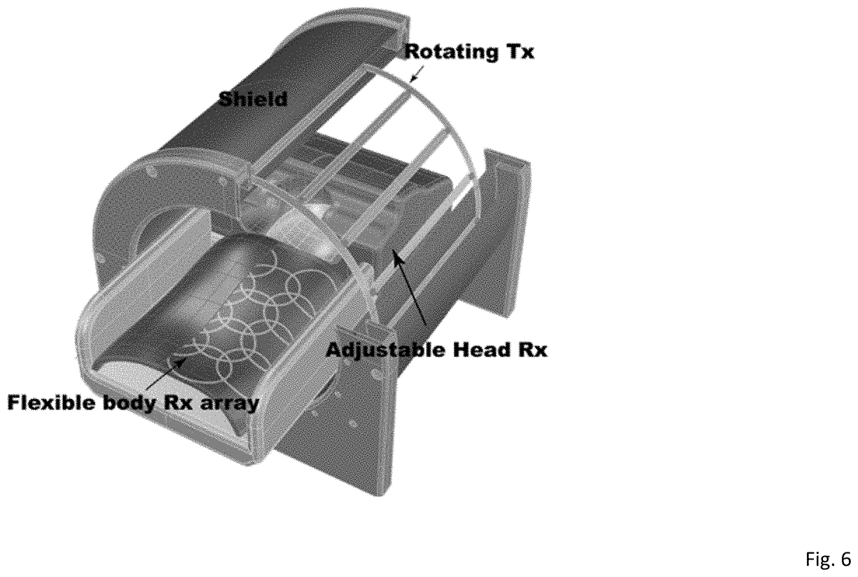

For interfacing the coil elements to an MR system, they can be connected to a small printed circuit board (PCB) of ˜20×25 mm. In some embodiments, the PCB can include a processor that interfaces with and/or controls the receive coil elements, a memory to store received data and operating instructions, etc. The interfacing PCB can be positioned between the two layers of textile of the wearable coil array. Polyethylene foam tape on the patient side of the PCB acts as a protective pad and ensures a distance of 1.5 mm for added protection against electric field, capacitive heating, and mechanical injury. Alternatively, a different protective pad (or component) may be used to protect the patient, such as a rubber pad, a silicon pad, solid foam, etc. On this coil interface board, nonmagnetic surface-mount capacitors (e.g., Knowls, Huntington, N.Y., USA) and inductors (e.g., Coilcraft Inc., Cary, Ill., USA) can be used to implement tuning, active and passive detuning, and matching. The latter is performed by a π network to achieve a double-peak frequency response for tolerance against wrapping and stretching. The matching network can then be connected to a low-impedance preamplifier module with input impedance of 2.5Ω at 64 MHz and a minimum noise figure of 0.6 dB. In some embodiments, a small correlation resistance can be included so that the noise figure increases slowly with deviation from optimum source impedance upon coil wrapping or changes in the load. In addition, the preamplifier, together with the 7E-matching network, complements approximate geometric decoupling of coil elements by preamplifier decoupling. FIG. 6 is a 3D illustration of the RPA pediatric coil system including a rotating transmitter coil, a 32-channel size adjustable head coil, a posterior cradle which houses 16 coil elements, and a flexible 16-channel blanket coil array in accordance with an illustrative embodiment.

In an illustrative embodiment, the receiver coil array(s) will be used with the rotating linearly polarized transmit coil (birdcage). The combined Tx/Rx coil system will be able to interface with both GE and Siemens MRI platforms. For this purpose, two separate interface boxes with the dedicated MRI system plugs can be designed, into which the constructed coil assembly will be plugged. In addition, two coil-to-patient-table footprints can be constructed that will allow the coil setup to be securely installed on the vendor's specific patient table.

It is worth noting that metal introduces both a susceptibility artifact due to B₀ distortion, and an RF artifact due to B₁ distortion from secondary magnetic fields generated by induced currents on the implant. The latter is particularly strong around elongated implants such as leads in active electronic devices and dominates the susceptibility artifact. The idea that the degree of distortion in B₁⁺ around a wire implant can be used to characterize (and in some cases, quantify) the RF currents induced in the wire, and by proxy its RF heating, has been used to reduce RF induced currents in guidewires using parallel transmit technique.

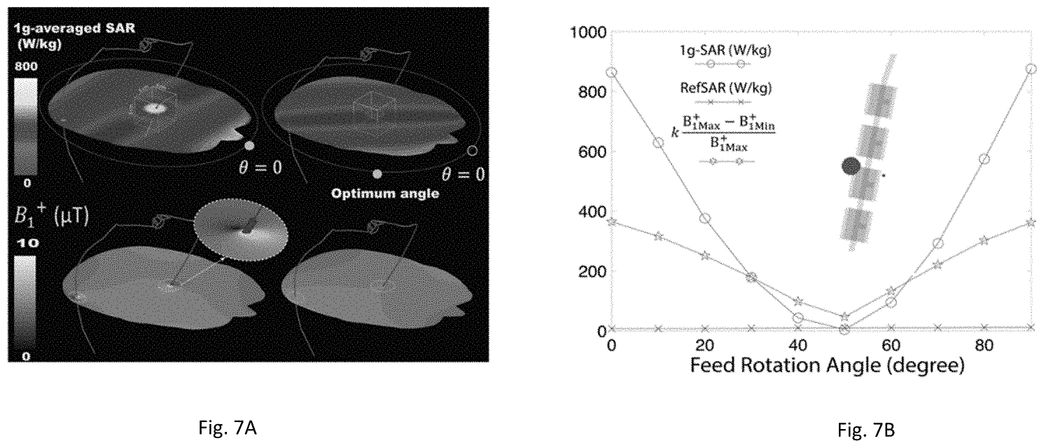

In a preliminary simulation study, the inventors demonstrated that an image-based marker which quantified the strength of visual distortion on B₁⁺ maps could successfully predict the optimum angle of a rotating coil to minimize local SAR around tips of a deep brain stimulation leads. This means that once the coil is positioned at a close-to-optimum angle for a certain device type (i.e., the statistically optimal angle θᵢ), a low-SAR fast B₁ mapping sequence can be applied to quickly fine-tune the coil for each individual. This technique can be used to quantify the performance of the RPA coil in an unseen cohort of patients, and to devise safety margins accordingly such that RF heating remains below 2° C. in the worst-case scenario. FIG. 7A is a simulation of a rotating head coil around a head phantom with a deep brain stimulation implant in accordance with an illustrative embodiment. FIG. 7B is an image-based metric to quantify the visual distortion in B₁⁺ maps (line with stars) perfectly correlated with the SAR amplification at a lead's tip in accordance with an illustrative embodiment. FIG. 7B suggests that minimizing the B₁ artifact would also minimize SAR.

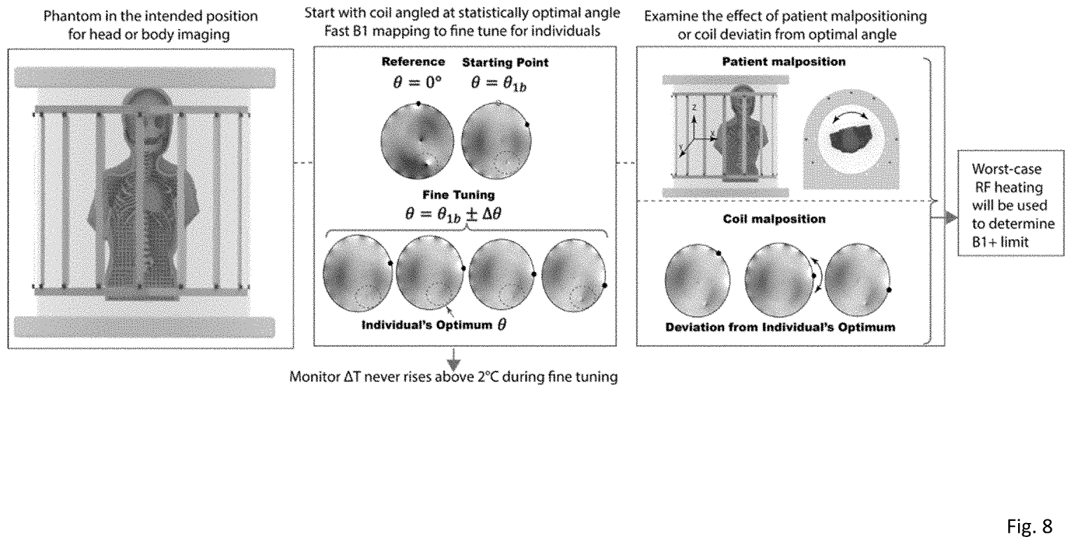

FIG. 8 is a diagram that depicts the proposed RPA imaging workflow in accordance with an illustrative embodiment. To validate the safety of the proposed workflow, 20 additional pediatric phantoms can be implanted with CIDEs (10, 5 epicardial), VNS devices (N=5), and EcoG systems (N=5). Temperature probes can be connected at multiple locations on lead and to the IPG for comprehensive monitoring. Phantoms can be positioned inside the RPA coil with the head or mid-chest at the iso-center. Testing can start with the RPA coil positioned at the statistically optimum angle based on the device type and imaging landmark. A fast low-SAR (<1 minute, B₁⁺<0.5 μT) B1 mapping sequence can be used to visualize the distortion around the implanted leads. The coil can also be rotated around this angle with 2° increments to find the position that minimizes the artifact while constantly monitoring the temperature rise ΔT during the fine-tuning process. Once the effective optimum angle is determined for each individual and at each imaging landmark, a sensitivity analysis is run to systematically disposition the phantom within the coil (1 cm in x, y and z directions, 5° rotation around z axis) to mimic patient motion, and deviate the coil by 10° from its optimum angle to mimic operator errors and run a high-SAR HASTE sequence (TE=99 ms, TR=2000 ms, FA=180, B1rms=3 μT, ΔT=10 minutes) to measure worst-case RF heating. The ΔT measured from this worst-case scenario can be used to determine the B₁⁺ limit for imaging that specific individual at that specific landmark as Equation 3 below.

$${B}_{1 limit}^{+} = 3 μ T \times \sqrt{\frac{2 ° C .}{Δ {T}_{worst - case}}} \qquad Equation 3$$

Using Equation 3, the proposed imaging workflow is considered validated for safety and efficiency responsive to determinations that 1) the ΔT recorded during the fine-tuning stage remains below 2° C. for all cases, and 2) the adjustment time to find the individual optimal angle remains <5 minutes. In alternative embodiments, different validation parameters may be used.

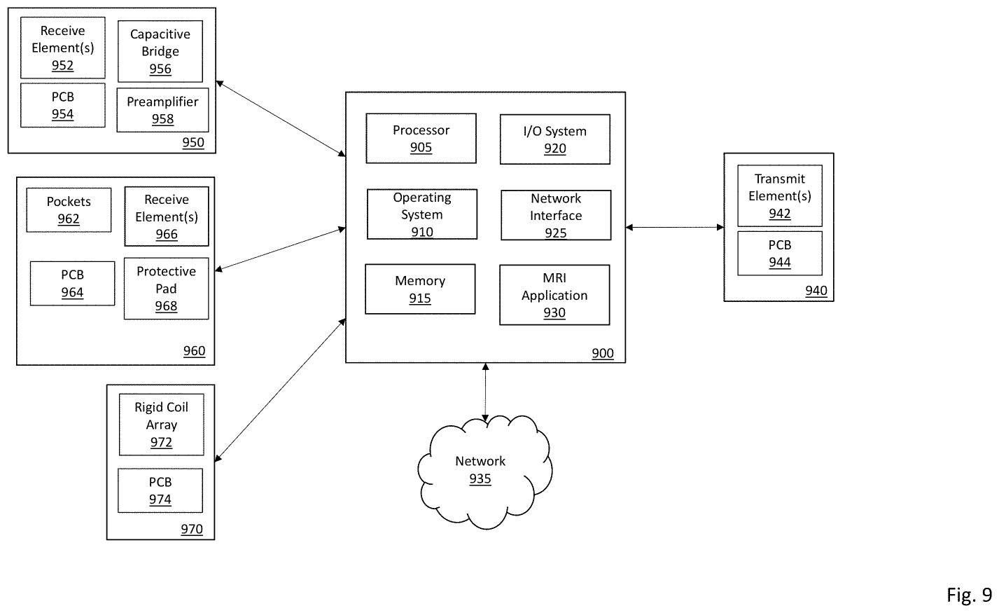

FIG. 9 is a block diagram of the proposed system in accordance with an illustrative embodiment. FIG. 9 depicts a computing device 900 in communication with a network 935. The computing device 900 is in direct or indirect (e.g., through the network 935) communication with a birdcage transmit coil 940, an adjustable head array 950, a blanket array 960, and a posterior cradle array 970. The computing device 900, which can act as a controller for the system, includes a processor 905, an operating system 910, a memory 915, an input/output (I/O) system 920, a network interface 925, and an MRI application 930. In alternative embodiments, the computing device 900 may include fewer, additional, and/or different components. In some embodiments, at least a portion of the computing device 900 is incorporated into one or more of the birdcage transmit coil 940, the adjustable head array 950, the blanket array 960, or the posterior cradle array 970. In some embodiments, each of the birdcage transmit coil 940, the adjustable head array 950, the blanket array 960, and the posterior cradle array 970 can include a separate computing device that includes elements of the computing device 900. In another embodiment, the computing device 900 can be independent of the birdcage transmit coil 940, the adjustable head array 950, the blanket array 960, and the posterior cradle array 970, and used to control the MRI system and its components.

The components of the computing device 900 communicate with one another via one or more buses or any other interconnect system. The computing device 900 can be any type of networked computing device. For example, the computing device 900 can be a tablet, a cell phone, a desktop computer, a dedicated device specific to the MRI application, etc.

The processor 905 can be in electrical communication with and used to control any of the system components described herein. The processor 905 can be any type of computer processor known in the art, and can include a plurality of processors and/or a plurality of processing cores. The processor 905 can include a controller, a microcontroller, an audio processor, a graphics processing unit, a hardware accelerator, a digital signal processor, etc. Additionally, the processor 905 may be implemented as a complex instruction set computer processor, a reduced instruction set computer processor, an x86 instruction set computer processor, etc. The processor 905 is used to run the operating system 910, which can be any type of operating system.

The operating system 910 is stored in the memory 915, which is also used to store programs, user data, network and communications data, peripheral component data, the task application 930, and other operating instructions. The memory 915 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 915 can be in the cloud to provide cloud storage for the system. Similarly, in one embodiment, any of the computing components described herein (e.g., the processor 905, etc.) can be implemented in the cloud such that the system can be run and controlled through cloud computing.

The I/O system 920 is the framework which enables users and peripheral devices to interact with the computing device 900. The I/O system 320 can include one or more displays (e.g., light-emitting diode display, liquid crystal display, touch screen display, etc.) that allow the user to view MRI settings and results, a speaker, a microphone, etc. that allow the user to interact with and control the computing device 900 and components connected thereto. The I/O system 920 also includes circuitry and a bus structure to interface with peripheral computing devices such as power sources, USB devices, data acquisition cards, peripheral component interconnect express (PCIe) devices, serial advanced technology attachment (SATA) devices, high definition multimedia interface (HDMI) devices, proprietary connection devices, etc.

The network interface 925 includes transceiver circuitry (e.g., a transmitter and a receiver) that allows the computing device to transmit and receive data to/from other devices such as the birdcage transmit coil 940, the adjustable head array 950, the blanket array 960, and the posterior cradle array 970, other remote computing systems, servers, websites, etc. The network interface 925 enables communication through the network 935, which can be one or more communication networks. The network 935 can include a cable network, a fiber network, a cellular network, a wi-fi network, a landline telephone network, a microwave network, a satellite network, etc. The network interface 925 also includes circuitry to allow device-to-device communication such as Bluetooth® communication.

The birdcage transmit coil 940 includes transmit element(s) 942 and a PCB 944. Alternatively, the birdcage transmit coil 940 may include fewer, additional, and/or different elements. Any type of transmitter known in the art may be used to implement the transmit element(s) 942). In an illustrative embodiment, the transmit elements 942 are linearly polarized. In an illustrative embodiment, the birdcage transmit coil 940 includes a plurality of ribs, and the transmit element(s) 942 are mounted to the ribs. The birdcage transmit coil 940 is mounted around a table such that the coil is able to bi-directionally rotate around at least a portion of the table. The PCB 944 can include any of the computer and/or circuitry components described herein, and can be used to control transmission by the transmit element(s) 942, rotation of the birdcage transmit coil 940, calculation of coil angles for the birdcage transmit coil 940, etc. In an alternative embodiment, any or all of the operations of the birdcage transmit coil 940 can be controlled by the computing device 900.

The adjustable head array 950 includes receive element(s) 952 mounted in an adjustable head cradle that is designed to receive the head of a patient. The receive element(s) 952 can be formed from copper wires with gaps cut therein to provide distributed capacitance in one embodiment. Alternatively, any other type of receive elements may be used. In another embodiment, the receive element(s) 952 are tiled in the adjustable head array 950 as an array of overlapping loops. A capacitive bridge 956 of the adjustable head array 950 is used to couple power out of the coil through critical coupling at a desired resistance (e.g., 50Ω). As discussed, the matching capacitor will additionally serve as part of an LC PIN diode trap circuit, designed to detune each receiver coil element during transmit. The capacitive bridge 956 and/or the trap circuit can be included on the PCB 954. The PCB 954 can also include a preamplifier 958 and any other circuit and/or computing elements described herein for use in controlling or implementing the adjustable head array 950. The preamplifier is used to reduce coupling between next-nearest and further neighbors in the coil array. In alternative embodiments, the adjustable head array 950 may include fewer, additional, and/or different elements.

The blanket array 960 includes pockets 962, a PCB 964, receive element(s) 966, and a protective pad 968. Alternatively, the blanket array 960 may include fewer, additional, and/or different elements. In an illustrative embodiments, the pockets 962 are formed with two layers of an elastic textile that are sewn together to create textile casings (or pockets) for embedding coil elements. Each of the pockets 962 is sized to receive a receive element 966. In an illustrative embodiment, the receive element(s) 962 are receive-only elements in the form of flexible coaxial cable, and are tiled in a 4×4 overlapped arrangement within the blanket array 960. In alternative embodiments, different configurations of the blanket array 960 can be used. The PCB 964 can include any of the circuitry and/or computing components described herein, and can be used to control operation of the blanket array 960. In one embodiment, the PCB 964 can be positioned between the two layers of textile that are used to form the pockets 962. The protective pad 968 is positioned between the PCB 964 and the patient for added protection against electric field, capacitive heating, and mechanical injury. The protective pad 968 can be a piece of foam tape, a rubber pad, a silicon pad, solid foam, etc.

The blanket array 960 is utilized to cover and protect the front side of a patient. The posterior cradle array 970, which includes a rigid coil array 972 and a PCB 974, is used to cover and protect a back side of the patient. In alternative embodiments, the posterior cradle array 970 may include fewer, additional, and/or different elements. The rigid coil array 972 can include a plurality of receivers (any type of receiver known in the art may be used) mounted in a rigid base. The PCB 974 can include any of the circuitry and/or computing components described herein, and can be used to control operation of the posterior cradle array 970.

The MRI application 930 can include software and algorithms in the form of computer-readable instructions which, upon execution by the processor 905, performs any of the various operations described herein such as predetermining an optimal coil position based on patient specific implant information, determining optimal coil angle(s), controlling a direction and amount of rotation of the birdcage transmit coil 940, controlling transmissions by the birdcage transmit coil 940, controlling signal reception by the adjustable head array 950, the blanket array 960, and the posterior cradle array 970, determining a region of zero-E field, steering the transmit coil 940 such that the region of zero-E field coincides with an implant, monitoring implant temperature with one or more temperature sensors, generating MRI images, etc. The MRI application 930 can utilize the processor 905 and/or the memory 915 as discussed above. In an alternative implementation, the MRI application 930 can be remote or independent from the user computing device 900, but in communication therewith.

Thus, described herein is a new MRI coil which has a region of low electric field, which can be mechanically rotated around the body of a patient such that an implanted cardiac (or other) lead is contained within the low-E field region. This use of a lower electrical field will significantly reduce RF heating of the implanted device, while not affecting the image quality at all. The proposed system is different from parallel transmit because instead of electronically modifying the shape of electric field for each patient, the proposed system mechanically rotates a coil with a predetermined electric field such that the implant of a patient is always positioned within a low-E field region of the coil. In an illustrative embodiment, the proposed system includes a linearly-polarized birdcage transmit coil which can mechanically be rotated around patient's body and adjusted in a patient-specific position such that patient's implant leads are contained within the coil's low-E field region. Such a system has been shown to have the potential to reduce RF heating of implants. Although the system has been described primarily with reference to use on infants and children, it is to be understood that the system is not so limited. In alternative implementations, the proposed system can be used on any patient (human or animal) to help minimize or eliminate RF heating.

Any of the methods described herein can be performed by a computing system that includes a processor, a memory, a user interface, transceiver, etc. Any of the operations described herein can be stored in the memory as computer-readable instructions. Upon execution of these computer-readable instructions by the processor, the computing system performs the operations described herein.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more”.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A system to perform magnetic resonance imaging, the system comprising:
a transmit coil that includes a plurality of transmitters, wherein the transmit coil is configured to receive at least a portion of an implant that is within a pediatric patient; and
a controller operatively coupled to the transmit coil, wherein the controller is configured to:
identify a region within the transmit coil with zero electric field while the transmitters are transmitting; and
rotate the transmit coil around the pediatric patient such that the implant is located within the region with zero electric field to avoid radio frequency heating of the implant.

2. The system of claim 1, wherein the plurality of transmitters are linearly polarized.

3. The system of claim 1, wherein the controller is further configured to determine, based on patient-specific information regarding a location of the implant within the pediatric patient, an optimal initial angle for the transmit coil.

4. The system of claim 1, wherein the transmit coil is in the form of a birdcage coil that includes a plurality of ribs, and wherein the transmitters are mounted on the ribs.

5. The system of claim 1, wherein the controller is configured to identify a plurality of angles of the transmit coil that result in the region with zero electric field.

6. The system of claim 1, further comprising an adjustable head array that includes a plurality of receivers and an adjustable head cradle that is configured to accommodate pediatric patients with varying head sizes.

7. The system of claim 6, wherein the plurality of receivers are formed from copper wires, and wherein the receivers receive signals from the plurality of transmitters in the transmit coil.

8. The system of claim 7, wherein each of the copper wires includes one or more gaps cut therein to provide distributed capacitance.

9. The system of claim 6, wherein the receivers are tiled in the adjustable head array as an array of overlapping loops.

10. The system of claim 6, wherein the adjustable head array includes a capacitive bridge to couple power out of the adjustable head array through critical coupling at a desired resistance.

11. The system of claim 6, wherein the adjustable head array includes a preamplifier to reduce coupling between neighboring receivers in the adjustable head array.

12. The system of claim 1, further comprising a blanket array that has a plurality of receive elements, wherein the blanket array is sized to cover a front portion of the pediatric patient.

13. The system of claim 12, wherein the receive elements are positioned in an overlapping arrangement within the blanket array.

14. The system of claim 12, wherein the receive elements are formed from flexible coaxial cable.

15. The system of claim 12, wherein the blanket array is formed from one or more textiles, and wherein the one or more textiles are arranged to include a plurality of pockets to receive the plurality of receive elements.

16. The system of claim 15, further comprising a printed circuit board positioned between layers of the one or more textiles.

17. The system of claim 16, further comprising a protective pad positioned between the printed circuit board and the pediatric patient.

18. A method of performing magnetic resonance imaging, the method comprising:
positioning a transmit coil that includes a plurality of transmitters about a pediatric patient such that the transmit coil receives at least a portion of an implant that is within the pediatric patient; and
identifying, by a controller, a region within the transmit coil with zero electric field while the transmitters are transmitting; and
rotating, by the controller, the transmit coil around the pediatric patient such that the implant is located within the region with zero electric field to avoid radio frequency heating of the implant.

19. The method of claim 18, further comprising identifying, by the controller, a plurality of angles of the transmit coil that result in the region with zero electric field.

20. The method of claim 18, further comprising determining, by the controller and based on patient-specific information regarding a location of the implant within the pediatric patient, an optimal initial angle for the transmit coil.
